(19) United States
(12) Patent Application Publication
(10) Pub. No.: US 2006/0286626 A1
(43) Pub. Date: Dec. 21, 2006

(54) Selective chromophores

(76) Inventors: Gunnar Westman, Harryda (SE); Eva Jennsiche, Goteborg (SE); Anders Hamberger, Onsala (SE)

Correspondence Address: GAUTHIER & CONNORS, LLP, 225 FRANKLIN STREET, SUITE 2300, BOSTON, MA, 02110, US

(21) Appl. No.: 11/005,269

(22) Filed: Dec. 6, 2004

Related U.S. Application Data
(63) Continuation of application No. PCT/SE03/00947, filed on Jun. 6, 2003.

(30) Foreign Application Priority Data
Jun. 6, 2002 (SE) 0201750-7

(51) Int. Cl. G01N 1/30; C07D 265/34

(52) U.S. Cl. 435/40.5; 544/99

(57) ABSTRACT

The present invention relates to chromophores for selective staining of cells or cell parts comprising a fluorescent chromophore of the general formulae I 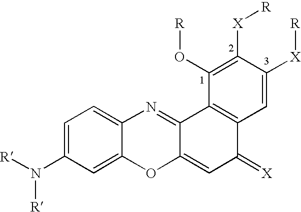 The invention further comprises a method for cell sorting, a method for cell targeting, as well as a method for cell identification.

### TECHNICAL FIELD

The present invention relates to chromophores for selective staining of cell and/or cell parts being a derivative of the so called Nile Red and Nile Blue compounds, as well as methods for sorting, identifying and targeting cells and cell parts, as well as intermediates for determining enzymatic activity in the cells.

### BACKGROUND

Fluorescent staining when combined with an appropriate imaging instrument is a sensitive method that is widely used in molecular biology and biochemistry laboratories. There are many commercially available fluorescent dyes but few operate in or near the far visible-near infrared region (600-1000 nm), which has some important advantages. The relative lack of specificity, of many dyes at the molecular level, stimulates the design of more selective dyes and staining methods.

The use of fluorescent dyes to identify different cells and detect surface molecules as well as proteins within cells has grown markedly in recent years. These dyes have become important markers and are included in one of the dominant visualizing techniques, fluorescence microscopy, due to its high sensitivity. Many commercially available fluorescent dyes are, however, limited by their lack of specificity such as low fluorescence. Since there are relatively few copies of most macromolecules present in any given cell and there are also tissues where one type of cells is rare, e.g. stem cells in testis, the detecting dyes have to be specific [1, 2].

U.S. Pat. No. 6,166,202 (=WO 97/29154) to Amersham Pharmacia Biotech UK Ltd relates to benzophenoxazine dyes, which are fluorescent and are able to label biomolecules. Some of the compounds disclosed are oxo carboxylic acids, oxo carboxylic acid esters, oxo ketocarboxylic acids, oxo ketocarboxylic acid benzoic esters, oxo carboxylic acid amide derivatives, and oxo-carboxylic acid-(diketo pyrrolidone esters). However, these dyes are not disclosed as being selective chromophores of cells to facilitate fractionating cell-study by chromophores. The above document does not disclose any of the present compounds, and is neither discussing selectivity.

U.S. Pat. No. 6,140,500 discloses benzophenoxazine nucleic acid dyes and method for their use. The derivatives are substituted in C7-NH2-position using propylammonium salts and relates to their binding to DNA.

Spiekermann et al., Archives of Microbiology, vol. 171, p 73-80, (1999) discloses a sensitive viable-colony staining method using Nile Red for direct screening of bacteria that accumulate polyhydroxyalkanoic acids and other lipids storage compounds. There is no disclosure of selective staining of cells.

In the early nineteenth century the demand for dyes to stain textiles led to a fertile period for organic chemistry. Some of the dyes were found to stain biological tissues and, unexpectedly, often showed a preference for particular parts of the cell.¹ Animal cells are not only tiny, they are also colourless and translucent, a variety of stains that provide sufficient contrast make those features visible. To day, dyes are used in a variety of applications and are of potential importance at least as diagnostic and therapeutic agents e.g. in the study of cancer. Cytochemical studies are essential to follow molecular mechanism involved in cellular pathways.
¹ Alberts, B., Bray, D., Lewis, J., Raff, M., Roberts, K., and Watson, J. D. (1994). How cells are studied. Molecular biology of the cell. (New York: Garland Publishing). pp 139-191.

Histology is the science of the microscopic structure of the tissues as well as how these individual components are connected. As most cells are colourless in their natural condition, they have to be stained in order to be able to become studied. During the last about one hundred years the knowledge has been available and has been developed. General practise is that the tissue material is stained using the dyes haematoxylin and Eosine. These two dyes have, however, the disadvantage of lacking enough high sensitivity for many studies. In particular, in those cases when one wants to study specific macromolecules of the cells. As these macromolecules are only available in a short number in the cell there will be no sensitivity high enough using many of the dyes available and used today. Furthermore, many of these dyes lack selectivity to specific cells or specific tissue materials. Thus it will often be necessary to carry out blocking steps, which block certain parts of the cells or whole cells. Using fluorescent dyes a higher selectivity will be reached and it will also be possible to study these said macromolecules and their reactions or the dynamics of the cell. In spite of the fact that more sensitive and selective dyes have been developed based on increased knowledge and understanding of how the chemical reactions take place and function within cells there is still a strong need for new dyes, which will bind selectively to specific cells.

Systems that work in the same way as the present compounds are available but these are manufactured in a considerably more complicated way than the compounds of the present invention. For example, GFP (green fluorescencing protein) conjugated to another protein, which binds to a specific site. The way these conjugates are prepared is described in i.a., the paper, The interaction of α-arrestin with the AP-2 adaptor is required for the clustering of β2-adrenergic receptor into Clathrin-coated pits., Caron et. al., The journal of Biological Chemistry vol. 275 pp. 23120-23126 (2000).

### SUMMARY OF THE PRESENT INVENTION

The present invention relates to chromophores for selective staining of cells and/or parts of cells being derivatives of the compounds Nile Red and Nile Blue. Thus it has been shown that certain derivatives in either of 1, 2, and 3 position are able to stain stem cell and spermatocytes in testis and all nucleuses in small intestine.

### DETAILED DESCRIPTION OF THE PRESENT INVENTION

It has now been found the compounds of the invention are selective chromophores for staining of cells which compounds comprises a fluorescent chromophore of the general formulae I 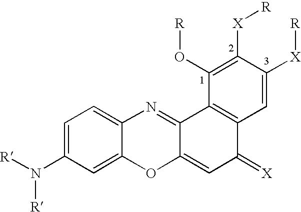
wherein only one of the positions 1, 2 or 3 is occupied by a group, wherein R is one of propinyl, aminoalkyl having 2 to 10 carbon atoms in the alkyl group, methoxy- (ethoxy)ₙ-alkyl having 2 to 10 carbon atoms in the alkyl group, and n being 0 to 4, (trimethylamino)-alkyl or (triethylamino)-alkyl or (tripropylamino)-alkyl or acetylamino alkyl having 2 to 10 carbon atoms in the alkyl group, (isoindolinyl-1,3-dione)-alkyl having 2 to 10 carbon atoms in the alkyl group and wherein X is O or N, and R′ is hydrogen, methyl, ethyl or propyl.

The propinyl group has its triple bond in end position.

Another aspect of the invention relates to colourless intermediates, of the formula II 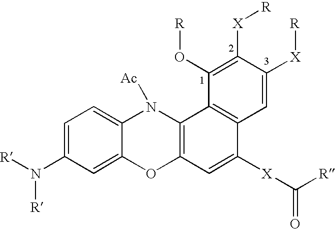
wherein R, n, R′ and X have the meanings given above, and R″ means lower alkyl, such as methyl, ethyl, propyl and butyl, or when X is N in 5 position, R″ can be an amino acid or a peptide sequence, which compounds upon interaction with an enzyme, such as an esterase or a phosphatase, are converted into the corresponding compound of formula I, wherein X in 5-position is O or N bound via a double bond to the aromatic nucleus. When a right R is resent in a right position the converted compound will enter its specific cell present, if present.

In those cases X is O in 5 position the intermediate compound can be of formula IIb 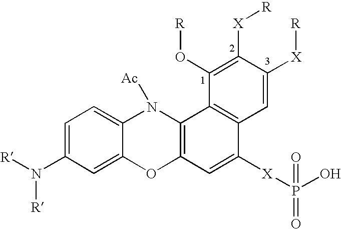
forming a phosphorous derivative. As indicated above the compounds of formula II and IIb can be used to show enzyme activity, such as by esterase, amidase, phosphatase and others. As given they are colourless but will obtain their characteristic colour at an enzymatic reaction.

The need for selective and fast staining methods stimulates the design of new fluorescent dyes. The dyes synthesized in this study, stain different cells depending on the position of the substituent and the substituent it self. PA-2 e.g. stains stem cells selectively, in testis from rat, compared to PASU-2, which stains spermatides and spermatozoa. In conclusion, there is a possibility to change the selectivity of dyes by relative small molecular changes. The cell staining is also dependent upon the concentration of dye added to the tissue.

The relative lack of specificity of many dyes at the molecular level stimulates the design of more selective fluorophores and staining methods. An excellent dye should operate in or near the far visible-near infrared region (600-1000 nm), have a large Stokes shift and chemical and photochemical stability. Molecules that absorb light in this region of the spectrum have advantages in that they show little susceptibility to optical interference from fluorescence of biological molecules, since most biological chromophores absorb and emit light at much lower wavelengths [3-5]. It is also a desirable operating region where the generation of radicals is minimized leading to a reduced risk of photobleaching and photo toxicity. In an aim to find new fluorophores for fast and selective staining methods we have investigated four substituted dyes based on the fluorophore Nile red. Nile red is a solvatochrome dye that is highly hydrophobic and binds to lipophilic parts. As a consequence it has mainly been used for lipid staining but also as a solvatochrome probe to measure the polarity of several liquids [6-8]. The phenoxazine dyes tend to have a large Stokes shift of 80-100 nm due to changes in the dipole moment and Nile red has the advantage as a highly fluorescent dye to absorb energy above 550 nm [9]. The new dyes were added to sections of testis from rat to investigate if the affinity could be altered by small molecular changes. Testis tissues were preferred because of its content of different cell types.

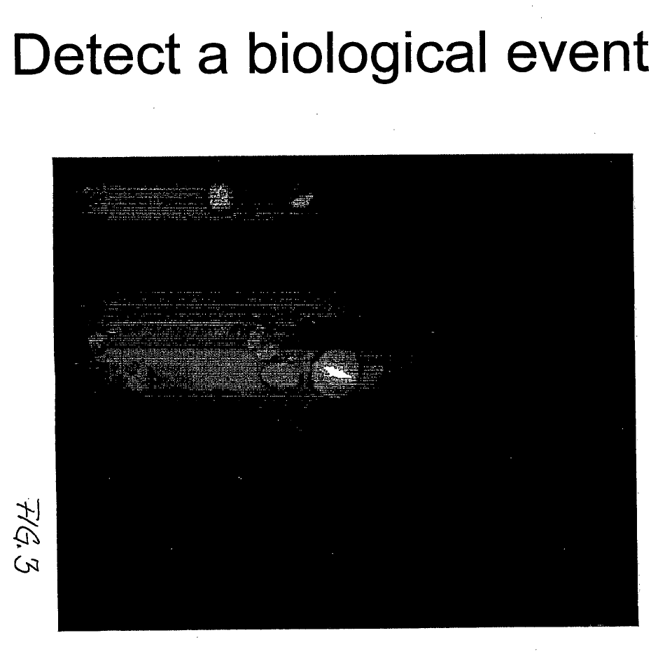

These dyes can also be used for studying events in the cell. As an example, mitosis, cell propagation, is shown in FIG. 3.

Further aspects of the invention encompass a method for cell sorting as to quality and quantity, a method for cell targeting, as well as a method for cell identification including determination of presence of intracellular structures or parts after a cell therapy or radiation therapy, such as after and during a cancer treatment.

A new class of red-emitting fluorescent benzophenoxazine dyes has been synthesized. These new dyes stain different cells. PA-2 is a very high fluorescent dye that stains stem cells and spermatocytes in testis and all nucleuses in small intestine. PASU-1 does not have any affinity compared to PASU-2 and PASU-3, which stain different cells and parts of cells.

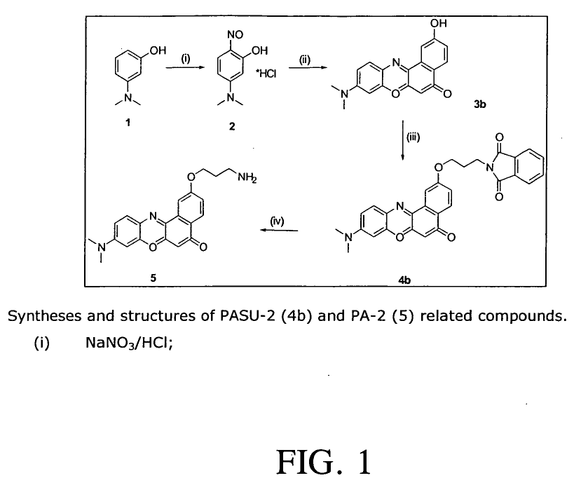

Selective staining of cells enables the possibility to sort cells so they can be studied individually. Since the study of stem cell biology is complicated due to the rare population of these cells in testis, possibly comprising as few as 2 in 10⁴ testis cells, development of methods for their enrichment are necessary [10, 11]. Formation of spermatozoa from germ cells is the result of the cytological events, known as spermatogenesis that takes place in the seminifreous tubule throughout the reproductive lifespan of the male (FIG. 1). This process is interrupted or subdivided into a series of distinct phases based on environmental cues that are transduced into hormonal signals stimulating or inhibiting spermatogenesis [12, 13]. There are three major phases first the stem cell renewal of the process of mitosis where the stem cells for the spermatogenic process are termed spermatogonia. The spermatogonia proceed to primary and secondary spermatocytes by entering meiosis, which is the second phase. In the third step the secondary spermatocytes divide by meiosis to form spermatides, which finally become mature spermatozoa. The spermatogenesis starts at the base of the tubule and the cells moves progressively toward the lumen of the tubule as the cells differentiate. This result in waves of reproductive activity which are achieved by the no dividing or stable population of sertoli cells [12]. There are signalling pathways that still have to be elucidated to understand the complete reaction that occurs in testis. Biochemical and molecular characteristics of spermatogonial stem cells have not been described, because a functional assay has not been available to unequivocally identify these cells [14, 15]. New opportunities would appear which may give enhanced understanding in e.g. reproduction problems that are quite common today, if the knowledge was not limited by unselective dyes, circumstance and time consuming staining methods.

There is a possibility to change the affinity of dyes by relative small molecular changes. The results also seem to be dependent upon the concentration of dye added to the tissue. Further more, the development and application of simple and selective fluorescent methods for routine detection of specific cells are of considerable interest since dyes are limited by their sensitivity.

Since the fluorophore/chromophore binds to specific parts in the cell it can be used in assays that monitor the movement of a target protein within a cell. Spotting foreign cells in skin, lung, thyroid or other tissue. This is usually difficult because they foreign cells don't look any different from their surroundings.

This invention describes the synthesis and shows a selective staining of different types of cells. As an example of the selectivity of these dyes, examples are shown how some of these dyes stain the same tissue material differently.

Pictures of How Different Cells are Stained

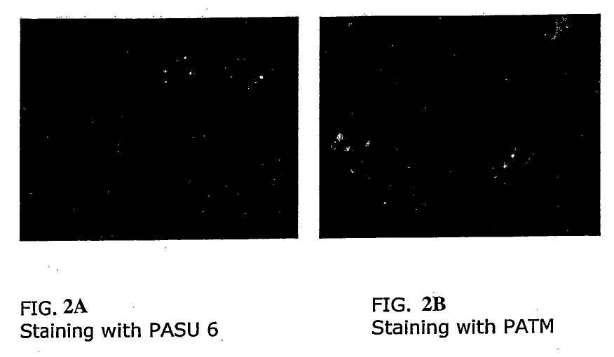

The compounds mentioned in the invention can be used for staining living cell cultures. An example is to identify receptors, which is clearly shown in FIGS. 2A and 2B. In FIG. 2A a specific binding within the cells is shown while FIG. 2B shows an almost non-specific binding, i.e., an even distribution of fluorescence.

As the receptor has not been stimulated the fluorescent marker will be evenly distributed in the cell, FIG. 2B. In FIG. 2A the activation of a receptor by the staining is shown, and the dye will only bind to the receptor. When the receptor becomes activated, which can take place by adding a further chemical entity; the fluorescent marker will bind preferably in the activated way. This has been made visible by the fact that only small points in the cell show fluorescence. Thus these compounds can be used for searching for known as well as unknown specific sites/receptors of proteins within cells. When the substituent of the fluorescent compound does not fit into any site the fluorescence will become evenly distributed within the cell, but if the substituent on the contrary, fits into a site this will result in that fluorescence is only shown as small points within the cell.

Since fluorescence microscopy only allows the wavelengths emitted from the dye, used to stain the section, it is a very sensitive instrument. By using fluorescent labels and fluorescence microscopy the sensitivity problem of the dyes was thought to be solved. The fluorescent dyes are also limited by its sensitivity and specificity, which depends on the dyes physical properties.² A successful fluorescent label must have a number of characteristics. It should have a large Stokes' shift, chemical and photochemical stability, and absorb light in the region 600-1000 nm. This absorption interval is a desirable operating region where there is minimum interference from fluorescence of biological molecules and reduced risk of photodecomposition. A large Stokes' shift is useful in simplifying the type of filter needed to differentiate between excitation and emission wavelengths, and may enable sensitivity limits to be improved.
² Nilsson, S. K., Hulspas, R., Weier H. U., and Quesenberry, P. J. (1996). In situ detection of individual transplanted bone marrow cells using FISH on sections of paraffin-embedded whole murine femurs. J Histochem Cytochem 44, 1069-1074.

(i) 1,6-dihydroxy naphthalene/DMF;
(iii) N-(3-bromopropyl)-phthalimide/K₂CO₃/DMF;
(iv) N₂H₄/EtOH/HCl. PASU-5 (4a) and 7 (4c) were synthesised in the same way with one exception, (ii) 1,5 and 1,7-dihydroxy naphthalene, respectively.

Derivatives of Nile Red (FIG. 1) were synthesized and their staining of testis and small intestine sections from rats were studied. Nile Red is a member of benzophenoxazine compounds that provide a family of fluorescent dyes, which can be used, for labelling biological molecules in various applications. The phenoxazine dyes tend to have a large Stokes' shift of 80-100 nm. Nile Red is a highly fluorescent dye, which absorbs energy above 550 nm. Consequently it has mainly been used for lipid staining but also as a solvatrochromic probe to measure the solvent strength of several liquids.

Results and Discussion

Synthesis

1-[3-(9-Dimethylamino-5-oxo5H-benzo[a]phenoxazine-2-yloxy)-propyl]-isoindole-1,3-dione (PASU-1),
b 2-[3-(9-Dimethylamino-5-oxo5H-benzo[a]phenoxazine-2-yloxy)-propyl]-isoindole-1,3-dione (PASU-2),
3-[3-(9-Dimethylamino-5-oxo5H-benzo[a]phenoxazine-2-yloxy)-propyl]-isoindole-1,3-dione (PASU-3) and
   2-(3-Amino-propoxy)-9-dimethylamino-benzo[a]phenoxazine-5-one (PA-2) were synthesized as outlined in FIG. 1 and in accordance with the examples given below. The phenol 2 was readily obtained by nitrosination of dimethylaminophenol 1 in hydrochloric acid [16]. Compound 2 was then condensed with 1,5, 1,6, and 1,7-dihydroxy naphthalene, respectively, to produce Nile Red derivatives 3a, 3b and 3c. These compounds were all substituted with N-(3-bromopropyl)-phthalimide to give PASU-1, 2 and 3. Derivative 3b was also reacted with trimethylammoniumpropylbromide to produce compound 4. PASU-2 was then converted into compound PA-2 via reduction with hydrazine.
   Staining of Testis and Small Intestine

In many areas of research including medical sciences, researchers and technicians often need to identify or sort cells. If the result can indicate the presence of a disease states, such applications require a fast, sensitive and selective method. The staining method developed for these dyes only include one incubation, the staining step, of 15 minutes. Compared to other staining processes in the field, which usually requires blocking step to obtain selectivity (72 hours or more), it is timesaving. Excitation and emission maxima of the synthesized compounds are in the red region of the visible spectrum which increase the sensitivity of fluorescence microscopy compared to the green region. Cellular components are generally transparent to red light and red lasers are also less expensive than the green lasers.

Significance

The specificity of dyes raises since it is relatively few copies of most macromolecules present in any given cell and more reaction cascades have to be elucidated to understand different diseases. Fluorescent dyes are more sensitive than non-fluorescent molecules but even these are limited. The affinity of many dyes available, at chemical basis, is not even known. We developed four fluorophores with small differences at the molecular level and studied them histologicaly. The dyes showed different characteristics in staining sections of testis and small intestine from rat. Compound 2-(3-Amino-propoxy)-9-dimethylamino-benzo[a]phenoxazine-5-one (PA-2) e.g. has high affinity for stem cells and spermatocytes in testis and all nucleuses in slices of small intestine. 2-[3-(9-Dimethylamino-5-oxo5H-benzo[a]phenoxazine-2-yloxy)-propyl]-isoindole-1,3-dione (PASU-2), another dye synthesized, has preferences to sperms and spermatides. The result differs a lot with the concentration of dye in the intestine, in this case. The compounds studied have large Stokes' shifts and absorb light at 550 nm, which are important characteristics of fluorescent dyes for biodetection.

The molecular affinity of these compounds is not known yet but it is interesting that even small molecular changes alter the specificity to different cells and parts of cells.

Staining of Human Fibro Sarcoma Cells

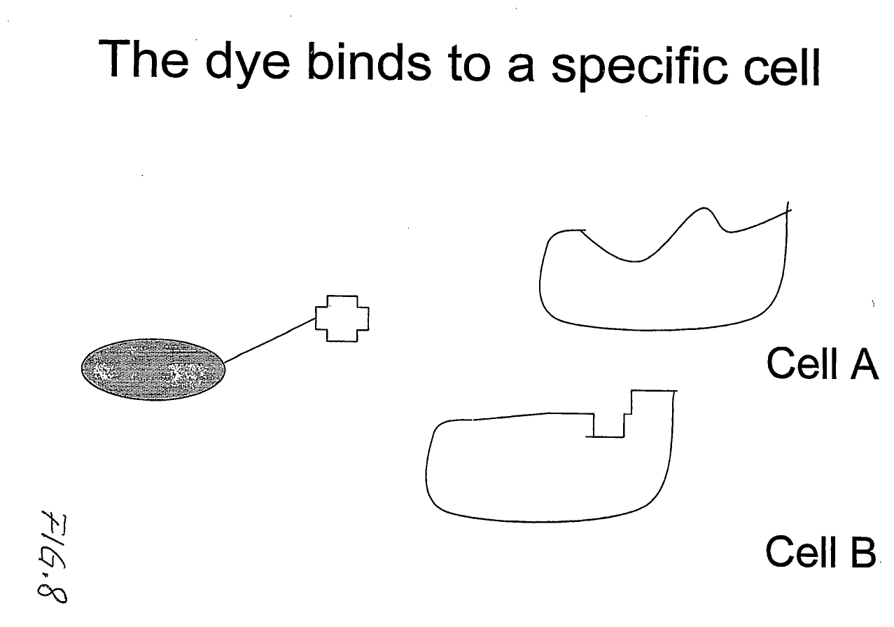

The dyes were also added to the human cell line of fibro sarcom, HT80, where PASU and PA showed great permeability of the plasma membrane compared to PATM-2, which stains the plasma membranes of the cells. The reason why PATM-2 does not reach into the cytoplasm of the cells is due to its cationic constituent. The positively charged part of the molecule might bind to the anionic phosphate group of phospholipids present in the plasma membrane. Since the PASU dyes are uncharged and relatively small molecules they have the ability to pass through the outer layer of the cells. FIG. 8 shows how PASU-2 stains the cytoplasm but not the nucleus in the cancer cells used in this study. To answer the question of what the dyes have affinity for further investigations have to be performed. Furthermore the dyes can be used for detection of receptors of cells. When the dye does not fit to the receptor it is evenly distributed throughout the cytoplasm. By adding a substance that induce conformational changes of the receptor which result in that the dye fit to the receptor, or use a dye with a substituent that fit in the inactivated receptor, the targets, receptors, can be detected as small fluorescent particles, so called pits. Thus, the dyes can be used to detect receptors and follow conformational changes of the receptor, activation-inactivation of receptor.

Materials and Methods

Equipment and Chemicals

Nuclear magnetic resonance (NMR) spectra were recorded on a Varian UNITY-VXR 500 spectrometer at 400 MHz, with deuterated dimethyl sulfoxide (DMSO) or chloroform (CDCl₃) with a few drops of DMSO added. Mass spectra were recorded on a Finnigan MAT TSQ 700 (San Jose, Calif.), using election impact (EI) as ionization technique. Chromatography was performed using silica gel (Merck, grade 60, 70-230 mesh, 60 Å). Reagents were purchased from Aldrich Chemical Company (Milwaukee, Wis.) and used without further purification. Fluorescence microscopic experiments were performed with a Zeiss microscope (West Germany) equipped with Plan Neofuar 40/0.9 I mm Ph3 and Plan Neofuar 25/0.8 W-Oel Ph2 objectives. A Winder M35 camera was used to obtain the microscopic images.

Synthesis of Compound 3:a, and b (Starting Materials)

5-dimethylamino-2-nitrosophenol hydrochloride (2.0 g, 10 mmol) and 1,5, 1,6, and 1,7-dihydroxynaphtalene (1.6 g, 10 mmol), respectively, were heated to 160° C. under reflux in dimethylformamide (DMF) (50 ml) for 4.5 h. The mixture was poured into water (400 ml) and the precipitate was collected by filtration. To increase the purity, the solid was refluxed in acetone for 10 minutes and stored at +8° C. overnight and filtrated. Only a small amount was obtained so a new attempt was made to precipitate more product from the filtrate by adding ethyl acetate and left at +8° C. overnight. The product was then collected by filtration (1.5 g, yield 49%).

Synthesis of PASU-1, 2 and 3 (Compound 4:a, b and c)

Compound 3:a and, b, respectively, (0.9 g, 3 mmol) and N-(3-bromopropyl)-phthalimide (0.8 g, 3 mmol) was added respectively to K₂CO₃ (8.3 g, 60 mmol) partly dissolved in dry dimethylformamide (30 ml). The mixture was stirred at 80° C. for 4.5 h and left in room temperature overnight. The reaction was completed according to thinlayer chromatography (TLC) in ethyl acetate. The product was precipitated in ethyl acetate (75 ml) and collected by filtration and washed with water and acetone to give the desired product (700 mg, yield: 47%). ¹H-NMR (CDCl₃): ¹H-NMR (CDCl₃), PASU-2: 2.18 (m, 2H), 3.11 (s, 6H), 3.90 (t, 2H), 4.22 (t, 2H), 6.13 (s, 1H), 6.45 (s, 1H), 6.68 (d, 1H), 6.94 (d, 1H), 7.56 (d, 1H), 7.71 (s, 2H), 7.80 (s, 2H), 7.90 (s, 1H), 8.04 (d, 1H). ¹H-NMR (CDCl₃), PASU-3: 2.26 (m, 2H), 3.14 (s, 6H), 3.97 (t, 2H), 4.22 (t, 2H), 6.41 (s, 1H), 6.54 (s, 1H), 6.72 (d, 1H), 7.14 (d, 1H), 7.64 (d, 1H), 7.66 (s, 1H), 7.74 (s, 2H), 7.86 (s, 2H), 8.54 (d, 1H).

Synthesis of PA-2 (Compound 5)

Compound 4c (0.6 g, 1.2 mmol) and N2H4 (0.1 g, 2.2 mmol) was added to ethanol (2 g) and heated to 90°. The reaction was followed by TLC in ethanol and completed after 1.5 h. HCl (0.3 ml, 5 mmol) was added to decompose hydrazine-hydrate and the reaction was continued another 0.75 h. The reaction was left in room temperature for 1 h before filtrated and washed with water. The filtrate was distilled to remove ethanol and med basic with excess of NaOH aq (20%). The product was extracted with CH2Cl2 (700 ml) and collected by evaporation. To purify the synthesized dye chromatography was performed. ¹H-NMR (CDCl₃), 2.15 (s, 2H), 3.20 (s, 6H), 4.10 (t, 2H), 4.11 (t, 2H), 6.42 (s, 1H), 6.57 (d, 1H), 6.96 (d, 1H), 7.47 (d, 1H), 7,83 (s, 1H), 8.01 (d, 1H), 8.13 (s, 1H), 8.13 (s, 2H)

Synthesis of PATM-3 (Compound 5′)

K₂CO₃ (0.8 g, 0.33 mmol) was partly dissolved in dry DMF (4 ml) and compound 3c (0.1 g, 0.33 mmol) and (3-brompropyl)trimethylammoniumbromide (0.09 g, 0.33 mmol) were added respectively. The mixture was heated to 80° C. and the reaction was completed after 3.5 h according to TLC in ethyl acetate. The product was precipitated in ethyl acetate (20 ml) filtrated and washed with acetone. ¹H-NMR: 2.35 (m, 2H), 3.05 (s, 6H), 3.25 (s, 9H), 3.55 (t, 2H), 4.30 (t, 2H), 6.25 (s, 1H), 6.75 (s, 1H), 6.85 (d, 1H), 7.30 (d, 1H), 7.65 (d, 1H), 8.00 (s, 1H), 8.10 (d, 1H).

In the same way the following compounds have been prepared
1-[3-(9-Dimethylamino-5-oxo-5H-benzo[a]phenoxazine-2-yloxy)-propyl]-isoindole-1,3-dione (PASU-1),
2-[3-(9-Dimethylamino-5-oxo-5H-benzo[a]phenoxazine-2-yloxy)-propyl]-isoindole-1,3-dione (PASU-2),
3-[3-(9-Dimethylamino-5-oxo-5H-benzo[a]phenoxazine-2-yloxy)-propyl]-isoindole-1,3-dione (PASU-3),
9-Dimethylamino-1-(trimethylammonium-propoxy)-benzo[a]phenoxazin-5-one (PATM-1)
9-Dimethylamino-2-(trimethylammonium-propoxy)-benzo[a]phenoxazin-5-one (PATM-2)
9-Dimethylamino-3-(trimethylammonium-propoxy)-benzo[a]phenoxazin-5-one (PATM-3)
1-(3-Amino-propoxy)-9-dimethylamino-benzo[a]phenoxazine-5-one (PA-1)
2-(3-Amino-propoxy)-9-dimethylamino-benzo[a]phenoxazine-5-one (PA-2)
3-(3-Amino-propoxy)-9-dimethylamino-benzo[a]phenoxazine-5-one (PA-3)
1-(3-propine-1-oxy)-9-dimethylamino-benzo[a]phenoxazine-5-one
2-(3-propine-1-oxy)-9-dimethylamino-benzo[a]phenoxazine-5-one
3-(3-propine-1-oxy)-9-dimethylamino-benzo[a]phenoxazine-5-one
   Staining

The stock solutions of the dyes were diluted with PBS or PBS+Triton-x to 5 μg/ml when added to both fixed and unfixed mature fibro sarcoma cells, cell line HT 80. Due to lack of time only the live cells untreated with Triton-x are discussed in the result.

Staining of Testis

Testis tissues were obtained from a male Sprague-Dawley rat, which has been used as a control in another study. The tissues were fixed in 4% formaldehyde, imbedded in paraffin and sectioned into 4 μm thick slices before dewaxed and hydrated. Stock solutions containing 5 mg dye/ml DMSO and ethanol were made. These were diluted to 0.05, 0.5, 5, 10, and 50 μg/ml with water. At the time of the experiment, 100 μl from each solution was added to the sections, respectively. The sections were incubated for 15 minutes before the excess dye was removed by washing with water.

In many areas of research including medical sciences, researchers and technicians often need to identify or sort cells. If the result can indicate the presence of disease states, such applications require a fast, sensitive and selective method. The staining method developed for the dyes in this report only includes one incubation, the staining step, of 15 minutes. The compounds synthesized absorb light around 550 nm and have emission maxima around 660 nm.

To se if and if so, the position of benzosuccinimidyl as a substituent alters the affinity of the dyes testis was stained with the two PASU dyes. PASU-2 binds to spermatides and spermatozoa which are closely related to each other since they are the only cells in testis that have a haploid number of single chromosomes. PASU-3 stains stem cells but not spermatides and spermatozoa. When staining with PA-2 the concentration of the dye added to the tissue determined which cells that were stained. At the lowest concentration, 0.05 μg/ml, only stem cells were stained, and when the amount of dye was increased to 0.5 μg/ml germ cells and spermatides fluoresced as well. When even higher content of PA-2 was used, 10 μg/ml, spermatocytes were stained as well. PATM-2 stains germ cells but show no concentration dependence.

Significance

Fluorescent staining when combined with an appropriate imaging instrument is a sensitive method that is widely used. Unfortunately many of the commercially available dyes are limited due to their absence of specific affinity. Immunochemistry is a powerful tool in molecular histology which includes antibodies and fluorophores but also preparations for non-specific binding. Immunological staining has the disadvantage that is time consuming due to incubations. Both the saving of time and the specificity of dyes can be solved by new molecular structures, which can be used by simple staining methods. We developed four fluorophores with small differences at the molecular level and studied them histologicaly. The compounds studied have large Stokes' shifts and absorb light at 550 nm, which are important characteristics of fluorescent dyes. Different staining patterns were obtained when staining sections of testis from rat. The compound 2-(3-Amino-propoxy)-9-dimethylamino-benzo[a]phenoxazine-5-one (PA-2) e.g. has affinity for stem cells at low concentrations in testis. The dyes synthesized show that relative small molecular changes contribute to totally different staining patterns. The molecular affinity of these compounds is not known but is in progress.

Fluorescence Microscopy and Image Processing

Fluorescence microscopic experiments were performed with a Zeizz (West Germany) microscope equipped with Plan Neofuar 40/0.9 I mm Ph3 and Plan Neofuar 25/0.8 W-Oel Ph2 objectives. A Winder M35 camera was used to obtain the microscopic images.

The results of selective staining are evident from the accompanying photos as indicated above.

Toxicology

The compounds are believed to be cytotoxic following intercalation into duplex DNA. Conventional wisdom believes that the effect is based upon the blocking of transcription for the duration of time that the intercalating agent is in place. Hydrophilic residues lying on the opposite side of the cytotoxic molecule, and thus exposed to the exterior of the complex, are positioned to code for termination of transcription. This causes the RNA polymerase to dissociate at the point and leave behind an incomplete and non-functional polymer.

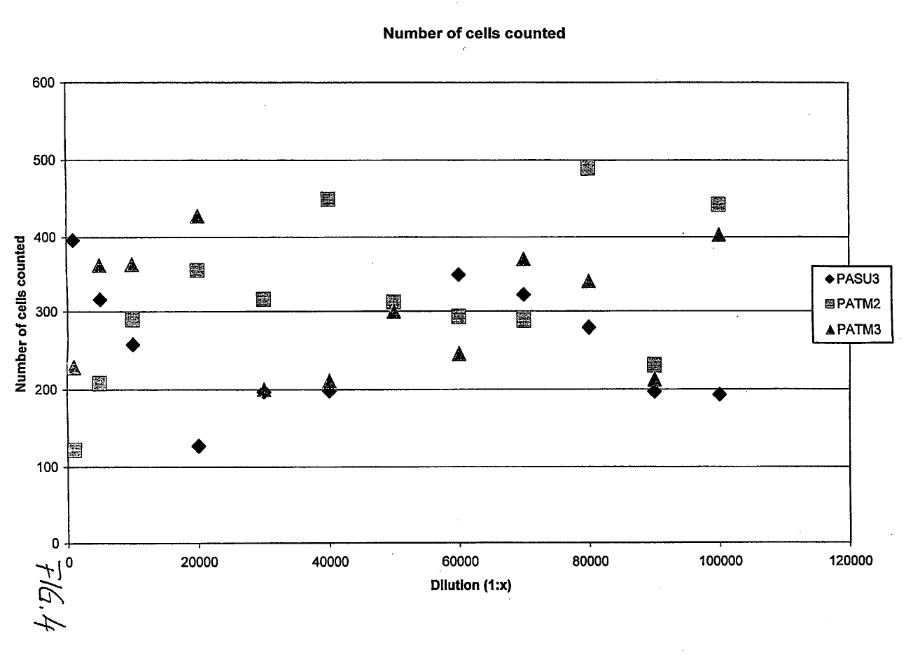

Toxicity is shown in FIG. 4, which is a graph over number of cells counted versus dilution.

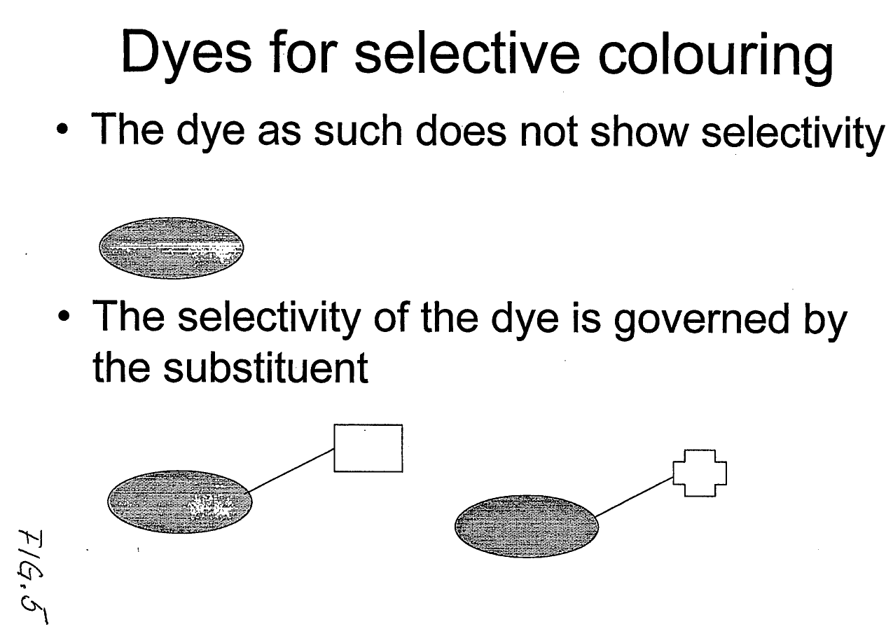

FIGS. 5-10 show the principle of the present invention, as well as some results from the dyeing of testis cells at spermatogenesis.

FIG. 5 shows the principle that the dye as such is not providing selective colouring, but the substituents of the dye provide the selectivity.

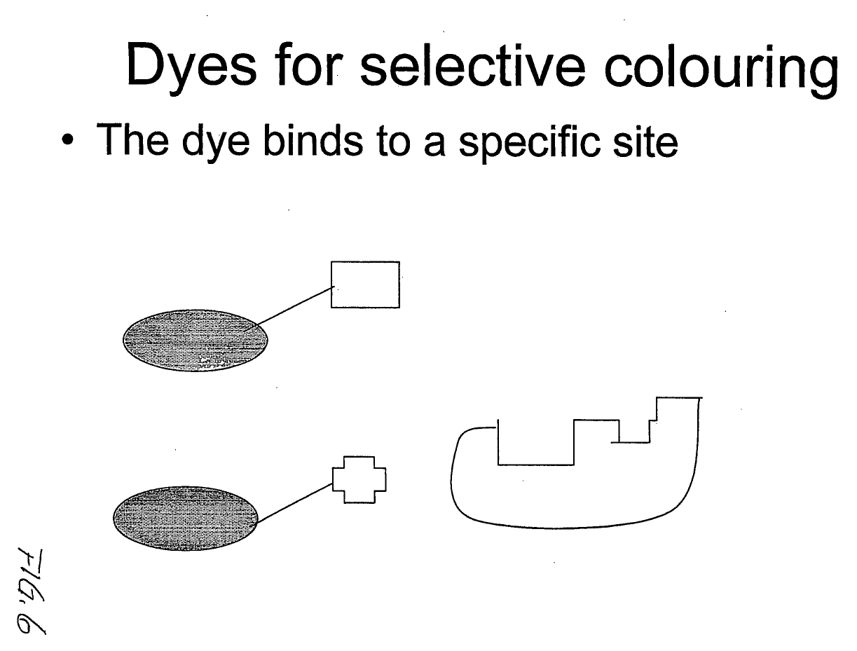

FIG. 6 shows the reason for selectivity, viz binding to a certain site, receptor site.

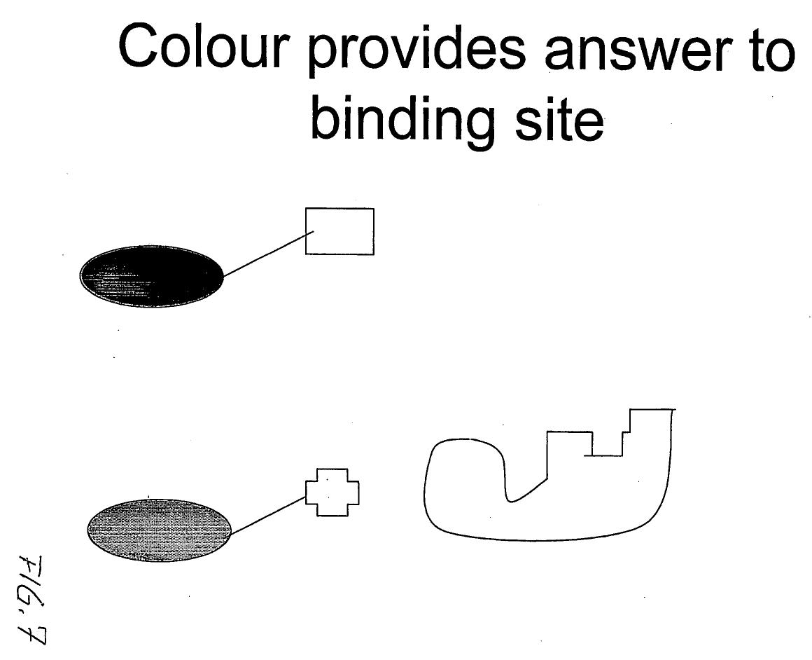

FIG. 7 shows differences in answer due to type of binding site.

FIG. 8 shows differences in answer when biding to cells, as they provide different binding sites.

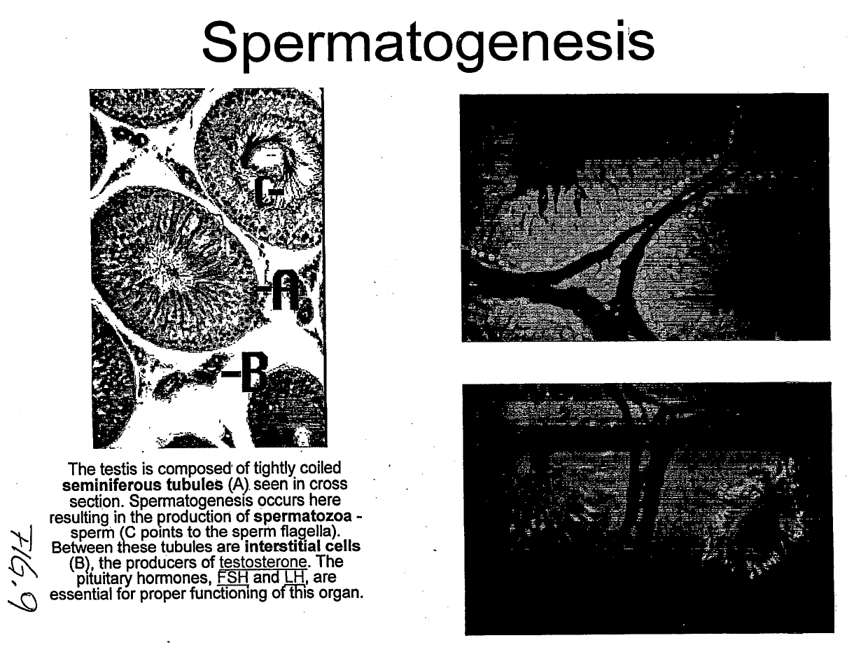

FIG. 9 shows spermatogenesis, wherein to the right of the lower picture the formation of sperms from the stem cell is shown, to the left of the lower picture shows the sperms during transfer to the cell surface, and the upper picture shows the sperm when they have migrated to the surface of the testis cells, and

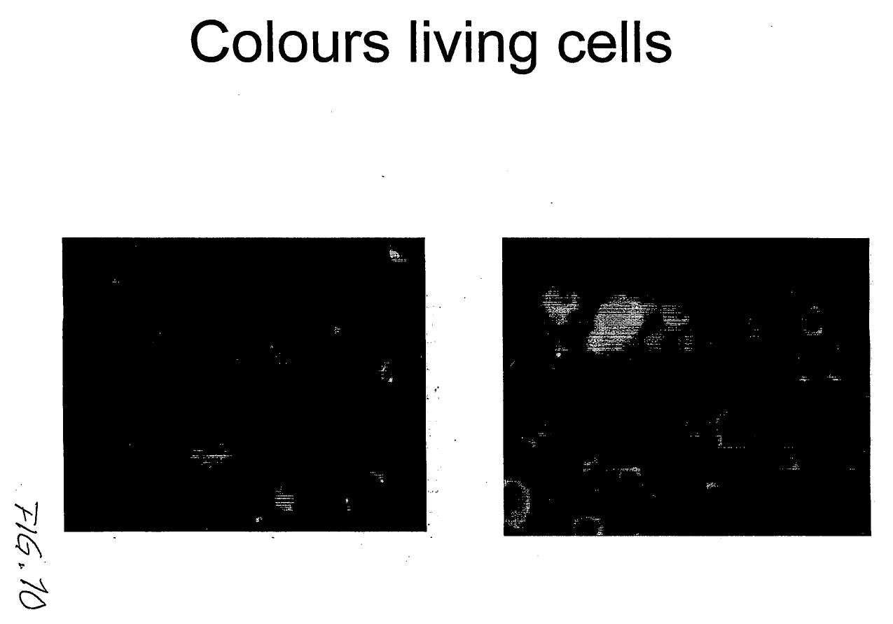

FIG. 10 shows colouring of living cells.

### REFERENCES

1. Alberts B., Bray D., Lewis J., Raff M., Roberts K., Watson J. D. (1989). How cells are studied. In Molecular biology of the cell, eds. (New York: Garland Publishing). pp 135-200.
2. Leblond C. P., Clermont Y. (1952). Definition of the stages of the cycle of the seminiferous epithelium in the rat. Ann. N.Y. Acad. Sci. 55, 548-573
3. Briggs M. S. J., Bruce I., Miller J. N., Moody C. J., Simmonds A. C., Swan E. (1997). Synthesis of functionalised fluorescent dyes and their coupling to amines and aminoacids. J. Chem. Soc. 1, 1051-1058
4. Patonay G., Antoine M. D. (1991). Near-infrared fluoregenic labels: New approach to an old problem. Anal. Chem. 63, 321A-327A
5. Klonis N., Wang H., Quazi N. H., Casey J. L., Neumann G. M., Hewish D. R., Hughes A. B., Deady L. W., Tilley L. (2001). Characterization of a series of far red absorbing perylene diones; A new class of fluorescent probes for biological applications. Journal of fluorescence. 11, 1-11
6. Greenspan P., Fowler S. D. (1985). Spectrofluorometric studies of the lipid probe, nile red. J. Lipid. Res. 26, 781-789
7. Deye J. F., Berger T. A., Andersson A. G. (1990). Nile red as a solvatochromic dye for measuring solvent strength in normal liquids and mixtures of normal liquids with supercritical and near critical fluids. Anal. Chem. 62, 615-622
8. Gressner A., Olbrich C., Schröder W., Kayser O., Müller R. H. (2000). The role of plasma proteins in brain targeting; species dependent protein adsorption patterns on brain-specific lipid drug conjugate (LCD) nanoparticles. Int. J. Pharm. 214, 87-91
9. Ghoneim N. (1999). Photophysics of Nile red in Solution Steady State Spectroscopy. Spectrochim. Acta. A. Mol. Biomol. Spectrosc. 56, 1003-1010
10. Shinohara T., Avarbock M. R., Brinster R. L. (1999). b₁- and a₆-integrin are surface markers on mouse spermatogonial stem cells. Proc. Natl. Acad. Sci. USA. 96, 5504-5509
11. Tegelenbosch R. A., Rooij D. G.d. (1993). A quantitative study of spermatogonial multiplication and stem cell renewal in the C3H/101 F1 hybrid mouse. Mutat. Res. 290, 193-200
12. Kretser D. M.d., Kerr J. B. (1994). The cytology of the testis. In The Physiology of Reproduction, Knobil E., Neill J. D. eds. (New York: Raven Press). pp 1177-1273.
13. Lincoln G. A. (1989). Seasonal aspects of testicular function. In The Testis, Burger H. G., Kretser D. M.d. eds. (New York: Raven Press). pp 329-385.
14. Chiang H.-S., Yeh S.-D., Lee W.-S. (2000). Spermatogonial stem cell culture and transplantation. New Taipei Journal of Medicine. 149-153
15. Kierszenbaum A. L. (1994). Mammalian spermatogenesis in vivo and in vitro; a partnership of spermatogenic and somatic cell line eages. Endocr. Rev. 15, 116-134
16. Crossley M. L., Dreisbach P. F., Hofmann C. M., Parker R. P. (1952). Chemotherapeutic dyes. I. 5-Aralkylamino-9-alkylaminobenzo[a]phenoxazines. J. Am. Chem. Soc. 74, 573-586
17. Chen Q.-y., Li D.-h., Zhao Y., Yang H.-h., Shu Q.-z., Xu J.-g. (1999). Interaction of a novel red-region fluorescent probe, Nile Blue, with DNA and its application to nucleic acids assay. Analyst. 124, 901-906
18. Huang C. Z., Li Y. F., Zhang D. J., Ao X. P. (1999). Spectrophotometric study on the supramolecular interactions of nile blue sulphate with nucleic acids. Talanta. 49, 495-503
19. Yang Y.-I., Hong H.-Y., Lee I.-S., Bai D.-G., Yoo G.-S., Choi J.-K. (2000). Detection of DNA using a visible dye, Nile Blue, in electrophoresed gels. Anal. Biochem. 280, 322-324
20. Karlsson H. J., Lincoln P., Westman G. (2002). Synthesis and DNA binding studies of a new asymmetric cyanine dye binding in the minor groove of [poly(dA-dT]₂. Bioorg. Med. Chem.

1. A chromophores for selective staining of cells or parts comprising a fluorescent chromophore of the general formulae I 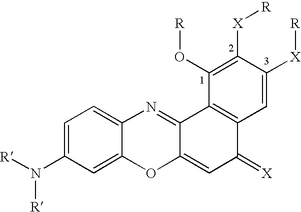
wherein only one of the positions 1, 2 or 3 is occupied by a group, wherein R is one of propinyl, aminoalkyl having 2 to 10 carbon atoms in the alkyl group, methoxy-(ethoxy)ₙ-alkyl having 2 to 10 carbon atoms in the alkyl group, and n being 0 to 4, (trimethylamino)-alkyl or (triethylamino)-alkyl or (tripropylamino)-alkyl or acetylamino alkyl having 2 to 10 carbon atoms in the alkyl group, (isoindolinyl-1,3-dione)-alkyl having 2 to 10 carbon atoms in the alkyl group and wherein X is O or N, and R′ is hydrogen, methyl, ethyl or propyl.

2. A compound according to claim 1, wherein the alkyl groups of R has 3 to 4 carbon atoms and R′ is methyl.

3. A compound according to claim 1, which is
1-[3-(9-Dimethylamino-5-oxo-5H-benzo[a]phenoxazine-2-yloxy)-propyl]-isoindole-1,3-dione (PASU-1),
2-[3-(9-Dimethylamino-5-oxo-5H-benzo[a]phenoxazine-2-yloxy)-propyl]-isoindole-1,3-dione (PASU-2),
3-[3-(9-Dimethylamino-5-oxo-5H-benzo[a]phenoxazine-2-yloxy)-propyl]-isoindole-1,3-dione (PASU-3),
9-Dimethylamino-1-(trimethylammonium-propoxy)-benzo[a]phenoxazin-5-one (PATM-1)
9-Dimethylamino-2-(trimethylammonium-propoxy)-benzo[a]phenoxazin-5-one (PATM-2)
9-Dimethylamino-3-(trimethylammonium-propoxy)-benzo[a]phenoxazin-5-one (PATM-3)
1-(3-Amino-propoxy)-9-dimethylamino-benzo[a]phenoxazine-5-one (PA-1)
2-(3-Amino-propoxy)-9-dimethylamino-benzo[a]phenoxazine-5-one (PA-2)
3-(3-Amino-propoxy)-9-dimethylamino-benzo[a]phenoxazine-5-one (PA-3)
1-(3-propine-1-oxy)-9-dimethylamino-benzo [a]phenoxazine-5-one
2-(3-propine-1-oxy)-9-dimethylamino-benzo[a]phenoxazine-5-one
3-(3-propine-1-oxy)-9-dimethylamino-benzo[a]phenoxazine-5-one

4. Compounds for selective stainin of cells comprising a compound of the formula I 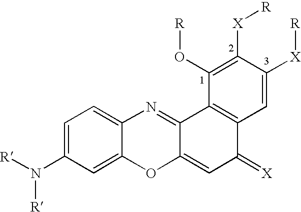
wherein only one of the positions 1, 2 or 3 is occupied by a group, wherein R is one of propinyl, aminoalkyl having 2 to 10 carbon atoms in the alkyl group, methoxy-(ethoxy)ₙ-alkyl having 2 to 10 carbon atoms in the alkyl group, and n being 0 to 4, (trimethylamino)-alkyl or (triethylamino)-alkyl or (tripropylamino)-alkyl or acetylamino alkyl having 2 to 10 carbon atoms in the alkyl group, (isoindolinyl-1,3-dione)-alkyl having 2 to 10 carbon atoms in the alkyl group and wherein X is O or N, and R′ is hydrogen, methyl, ethyl or propyl.

5. A method for sorting and/or counting cells by using a selective chromophore, whereby the cells to be sorted are stained with one or more of the compounds according to claim 1.

6. A method according to claim 5, wherein qualitative sorting of cells is carried out.

7. A method according to claim 5, wherein quantitative sorting of cells is carried out.

8. A method for targeting selective cells, whereby cells to be targeted are stained using one or more of the selective chromophores according to claim 1.

9. A method for identification of cells, whereby the cells to be identified are selectively stained using one or more selective chromophore according to claim 1.

10. A method according to claim 9, wherein cells are identified as to presence of structure after a cell therapy.

11. A method according to claim 9, wherein cells are identified as to presence and/or structure after radiation treatment.

12. Intermediate compounds for the manufacture of chromophores of claim 1 for determining intracellular reactions having the formula II 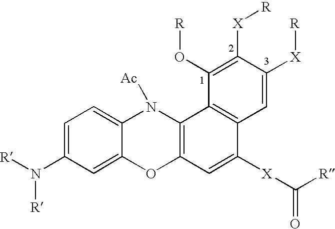
or of formula IIb when X in 5 position is O 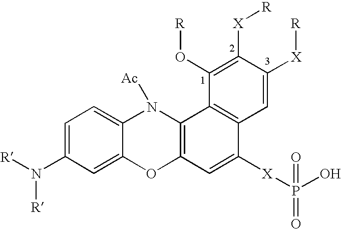
wherein only one of the positions 1, 2 or 3 is occupied by a group, wherein R is one of propinyl, aminoalkyl having 2 to 10 carbon atoms in the alkyl group, methoxy-(ethoxy)ₙ-alkyl having 2 to 10 carbon atoms in the alkyl group, and n being 0 to 4, (trimethylamino)-alkyl or (triethylamino)-alkyl or (tripropylamino)-alkyl or acetylamino alkyl having 2 to 10 carbon atoms in the alkyl group, (Isoindollnyl-1,3-dione)-alkyl having 2 to 10 carbon atoms in the alkyl group and wherein X is O or N,R′ is hydrogen, methyl, ethyl or propyl and R″ is lower alkyl, preferably methyl, ethyl or propyl, or when X in 5 position is N, R″ can be an amino acid or a peptide sequence having the ability of being transformed into a compound of formula I when made subject to an enzyme acting on the O-acetyl or N-acetyl group forming the corresponding compound of formula I wherein X is O or N in 5-position.
